(19) United States
(12) Patent Application Publication
(10) Pub. No.: US 2022/0249800 A1
(43) Pub. Date: Aug. 11, 2022

(54) STIMULATING AN AGE REGRESSION EXPERIENCE

(71) Applicants: Garland Frank Lawlis, Sanger, TX (US); Thurman Frank Lawlis, Celina, TX (US)

(72) Inventors: Garland Frank Lawlis, Sanger, TX (US); Thurman Frank Lawlis, Celina, TX (US)

(21) Appl. No.: 17/419,168

(22) PCT Filed: Feb. 11, 2021
(86) PCT No.: PCT/US2021/017531

§ 371 Date: Jun. 28, 2021

(51) Int. Cl. A61M 21/02

(52) U.S. Cl. CPC A61M 21/02; A61M 2021/0027

(57) ABSTRACT

A method comprising stimulating an age regression experience in a human subject. Presenting, for the subject's attention, an audio relaxation program to induce a hypnogogic state in the subject and while the subject is in the hypnogogic state, presenting a visual stimulus and an audio stimulus corresponding to an earlier experience in the subject's life. An apparatus comprising an audio-video module to simulate an age regression experience in a human subject. The audio-video module includes an audio component to present an audio relaxation program to induce a hypnogogic state in the subject, and while the subject is in the hypnogogic state, present an auditory stimulus corresponding to an earlier experience in the subject's life and a video component to present a visual stimulus corresponding to the earlier experience.

### CROSS REFERENCE TO RELATED APPLICATION

Pursuant to 35 U.S.C. § 119(e), this application claims priority to U.S. Provisional Application Ser. No. 62/972,691, filed Feb. 11, 2020, the contents of which are hereby incorporated by reference in its entirety.

### TECHNICAL FIELD

This application is directed, in general, to stimulating an age regression experience in human subjects and, more specifically, to a method and apparatus to facilitate stimulating the age regression experience.

### BACKGROUND

Some observations suggest that a person's sense of age is related to the environment in which they live, and, that changing that living environment can change one's sense of age. Other observations suggest that listening to music can improve a person's psychological, emotional and intellectual state. Observations of Native American ritual practices suggest that such practices can shift a person's reality, by shifting one's self-concept to adopt a particular animal species' ability or attitude.

### SUMMARY

One embodiment of the disclosure is a method that comprises stimulating an age regression experience in a human subject. Stimulating the age regression experience can include presenting, for the subject's attention, an audio relaxation program to induce a hypnogogic state in the subject; and then, while the subject is in the hypnogogic state, stimulating the age regression experience can also include presenting, for the subject's attention, a visual stimulus and an audio stimulus corresponding to an earlier experience in the subject's life.

Another embodiment of the disclosure is an apparatus that comprises an audio-video module to simulate an age regression experience in a human subject. The audio-video module can include an audio component to present for the subject's attention, an audio relaxation program to induce a hypnogogic state in the subject, and then, while the subject is in the hypnogogic state, can present for the subject's attention, an auditory stimulus corresponding to an earlier experience in the subject's life. The audio-video module can also include a video component to present for the subject's attention, a visual stimulus corresponding to the earlier experience.

### BRIEF DESCRIPTION

The embodiments of the disclosure are best understood from the following detailed description, when read with the accompanying FIGUREs. Some features in the figures may be described as, for example, “top,” “bottom,” “vertical” or “lateral” for convenience in referring to those features. Such descriptions do not limit the orientation of such features with respect to the natural horizon or gravity. Various features may not be drawn to scale and may be arbitrarily increased or reduced in size for clarity of discussion. Reference is now made to the following descriptions taken in conjunction with the accompanying drawings, in which:

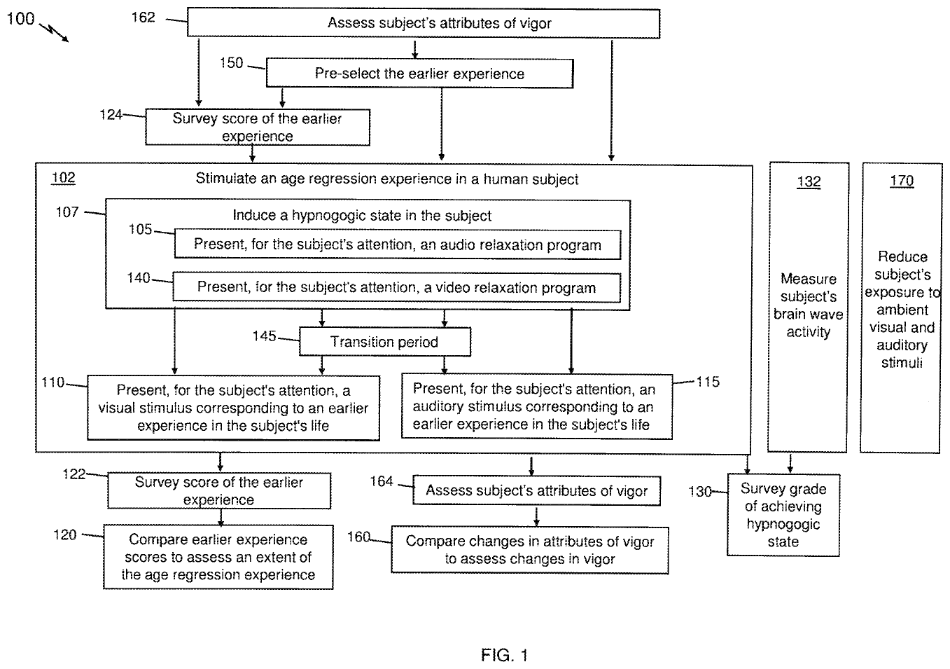

FIG. 1 presents a flow diagram illustrating example embodiments of a method to stimulate an age regression experience in accordance with the disclosure; and

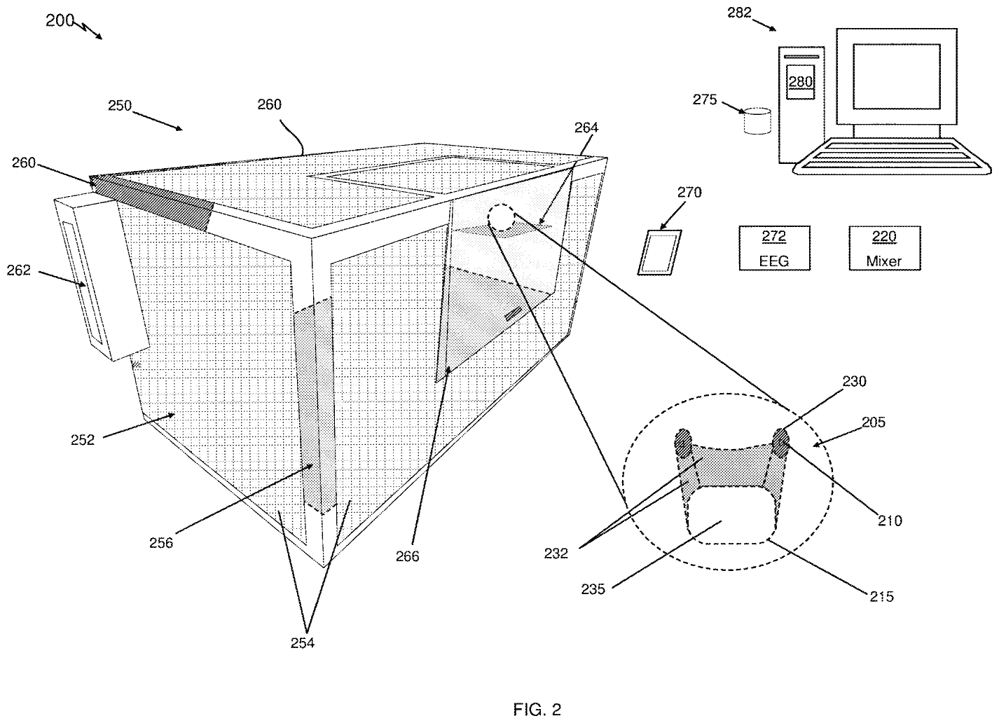

FIG. 2 presents a perspective view of an example apparatus, such as implemented by any of the embodiments of the method disclosed in the context of FIG. 1, to simulate the age regression experience.

### DETAILED DESCRIPTION

Embodiments of the present disclosure benefit from our recognition of three general experiential principles and their integration, to facilitate a subject's age regression to an earlier experience in their life: 1) an environment principle: exposing a human subject to a visual environment reflective of an earlier experience in that subject's life can help shift the subject's self-image to an earlier self-image similar to a self-image the subject had during that earlier experience; 2) an auditory principle: exposing the subject to an audio environment reflective of that same earlier experience can further help the shift the subjects' self-image to the earlier self-image; 3) a reality-shifting principal: presenting a relaxation program to help the subject enter a hypnogogic state can facilitate the shift to the earlier self-image.

We believe that shifting the subject's self-image to an earlier self-image can stimulate an age regression experience, which in turn, can help the subject to regain a degree of vigor similar to the vigor that the subject experienced at the time of the original earlier experience. Consequently, the subject can have improved physical, emotional or intellectual abilities.

Some embodiments of our method and apparatus can facilitate a subject attaining the age regression experience within a short session (e.g., ½ to 2 hour). For instance, instead of having the subject change their living environment, e.g., by dressing and living in an actual environment portraying the earlier experience for days or weeks, our method and apparatus permits the subject to be presented with images and sounds representative of the earlier experience in a virtual reality environment. For instance, instead having the subject participate in a ritual practice for several hours, or days, to attain an animal identity, the subject is presented with a relaxation program to help the subject rapidly enter a hypnogogic state to facilitate regression to a former self-image identity corresponding to the earlier experience in that subject's life.

Embodiments of the method and apparatus disclosed herein help integrate the three experiential principles in a particular sequence to facilitate the rapid stimulation of the age regression experience, and thereby provide a more efficient use of the subject's time and the resources used to help stimulate the age regression experience.

One embodiment of the disclosure is a method. FIG. 1 presents a flow diagram illustrating example embodiments of the method 100 and FIG. 2 presents a perspective view of an example apparatus 200, e.g., to implement embodiments of the method.

With continuing reference to FIG. 1 throughout, the method 100 comprises stimulating an age regression experience in a human subject (step 102). Stimulating the age regression experience, e.g., as part of step 102, includes presenting, for the subject's attention, an audio relaxation program (step 105) to induce a hypnogogic state in the subject (step 107). Stimulating the age regression experience also includes, presenting (e.g., after inducing the hypnogogic state in step 107) for the subject's attention, a visual stimulus (step 110) and presenting an auditory stimulus (step 115) corresponding to an earlier experience in the subject's life.

The term age regression experience, as used herein, refers to the subject's subjective enhanced recollection of the earlier experience after the stimulation is over.

In some embodiments of the method 100, for example, an extent of the subject's age regression experience during the stimulating (step 102) can be assessed by comparing (step 120) changes in the subject's self-assessment of the depth and the intensity the subject's recollection of the earlier experience made after the stimulating step 102 (step 122) to the subject's self-assessment of the depth and intensity the subject's recollection made before the stimulating step 102 (step 124). For instance, as part of self-assessment steps 122 and 124, the subject can complete a self-assessment survey, or be interviewed, to establish a score reflective of their age regression experience, and as part of step 120, a difference, or relative change, in the before and after scores can be calculated to evaluate the subject's enhanced recollection of the earlier experience due to the stimulating in step 102.

The term hypnogogic state, as used herein, refers a state of deep relaxation where a subject's state of consciousness is transitional between wakefulness and sleep, and where the perception of reality is fluid.

In some embodiments of the method, for example, the presence of the hypnogogic state during the stimulating step 102 can be assessed in step 130 by having the subject complete a survey, or by interviewing the subject, after the stimulating step 102, where the subject grades their state of relaxation and experiences of dream-like visions, sounds or other types of hypnagogic experiences achieved during the simulating step 102, including the presentation of the audio relaxation program (step 105) and/or during the subsequent presentations of the visual and audio stimuli (steps 110, 115).

Alternatively, or additionally, the presence of the hypnogogic state can be assessed by measuring the subject's brain wave activity during the stimulating (e.g., step 132 during step 102) and/or subsequent presenting of visual and auditory stimulus (e.g., step 132 during steps 110 and 115).

One skilled in the pertinent art would understand how to collect and interpret such brain wave measurements to determine the presence of a hypnogogic state. As a non-limiting example, as part of step 132 electrodes can be attached to multiple areas of the subject's scalp (e.g., scalp areas placed according to the international 10/20 system, or other standardized systems familiar to those skill in the pertinent art), and the subject's brain wave activity can be measured using equipment familiar to one skilled in the pertinent art, during the stimulating step 102. In some embodiments, for example, the hypnogogic state can be considered to have been achieved when one or more of the electrodes (or a majority of the electrodes, or all of the electrodes in various embodiments) records the presence of Theta (e.g., about 4 to 8 Hz) and/or Delta (e.g., about 1 to 4 Hz) waveform activity in an electroencephalogram (EEG) spectrum. For example when stimulating the age regression experience for the first time in a new subject, it can be advantageous to perform both the survey/interview (step 130) and brain wave activity measurement (step 132) to confirm that the hypnogogic state was achieved during step 105 and maintained during steps 110 and 115. Performing both the survey/interview and brain activity measurements facilitates correlating the former with the later to provide greater confidence in future assessments of confirming the presence of the hypnogogic state for subsequent stimulation sessions by using the survey/interview step 130 only.

We believe that it is important for the subject to enter the hypnogogic state, as induced by step 105, before presenting the visual or audio stimuli in steps 110 and 115 because the subject needs to be switched out of their present state of consciousness and self-image, so that they can shift to the earlier self-image associated with the earlier experience. We also believe that it is important for the subject to continue to be in the hypnogogic state throughout the duration of the visual or audio stimuli corresponding to an earlier experience so that the subject can gain feelings of vigor similar to what they felt at the time of the original experience.

In some embodiments, the presentation of the audio relaxation program to induce the hypnogogic state in step 105 can be for about 10 minutes (e.g., in a range from 5 to 15 minutes for some embodiments). In our experience, this is about the average time it takes for a subject to shift their state of consciousness. In some embodiments, the presentation of the visual and audio stimuli in steps 110 and 115 can be for about 60 minutes (e.g., in a range from 30 to 120 minutes for some embodiments). In our experience, this is about the minimum time that a subject can comfortably remain in the hypnogogic state while watching and listening to the visual and audio stimuli and thereby have the age regression experience. Longer periods (e.g., greater than 2 hours) of visual and audio stimuli in steps 110 and 115 can be possible for individuals that greatly enjoy the stimuli while still remaining in the hypnogogic state.

Embodiments of the audio relaxation program presented as part of step 105 can be or include a series of vocal relaxation instructions, e.g., words instructing the subject to progressively and sequentially to slow down and deepen their breathing, to relax their body parts (e.g., from feet to legs to trunk to arms to hands to neck and head) and to inwardly focus, until the hypnogogic state is induced. In some embodiments, the vocal relaxation instruction can be presented from a pre-recorded script of instructions. In some embodiments, such as when EEG measurements are made to assess or confirm the induction of the hypnogogic state, EEG biofeedback can be included as part of the audio relaxation program in step 105, e.g., in the form of audio tones, pulses or other sounds reflective of the subject's present state of brain wave activity.

In some embodiments, the method 100 can further include a video relaxation program (step 140) to facilitate inducing the hypnogogic state as part of step 107. Including the video relaxation program can advantageously prompt the subject to keep their eyes open so that they are primed to receive the subsequent presentation of the visual stimulus in step 110. As non-limiting examples, the visual relaxation program as part of step 140 can include moving images, presented on a view screen, of a flame, e.g., a fireplace a candle, an object, e.g., a circle or boat or cloud, travelling slowing across the subject's visual field, or melting ice, e.g., a melting ice cube.

In some embodiments of the method, the audio relaxation program (step 105), and the optional visual relaxation program (step 140), can be abruptly halted before starting the presentations of the visual stimulus and/or the auditory stimulus (steps 110, 115). In other embodiments, to help avoid disturbing the subject's hypnogogic state, the audio relaxation program and optional visual relaxation program can be gradually reduced in volume and intensity and then halted while the presentations of the visual stimulus or the auditory stimulus are gradually increased in intensity and volume. As a non-limiting example, in some embodiments, after inducing the hypnogogic state in step 102, a transition period (step 145, e.g., a 1, 2, or 5 minute period, for some embodiments) can follow where the audio and video relaxation programs are faded out to become non-audible and non-visible, respectively, while at the same time, the visual stimulus and the auditory stimulus are faded in to become visible and audible, respectively.

In some embodiments, the earlier experience can be or include an earlier era of time corresponding to a time period experienced by the subject, e.g., when the subject recollects feelings of vigor (e.g., physically, emotionally and intellectually healthy, stable and competent). The earlier experience can be or include an earlier sensation felt by the subject, e.g., where the subject recollect feelings of vigor, e.g., a sensation of experiencing the feel of mud, a warm bath, or the smell and taste dish of food.

In some embodiments the earlier experience can include experiences from both an earlier era of time and an earlier sensation.

Non-limiting example embodiments of the visual stimulus presented in step 110 can include presenting one of more of image copies of pictures, drawings, photographs or video recordings produced in a time period corresponding to the earlier experience. For instance, when the earlier experience corresponds to an earlier era in the subject's life, the image copies can be from publically available records, and/or the subject's personal recordings, of pictures, drawings, photographs or video recordings produced during the earlier era. For instance, when the earlier experience corresponds to an earlier sensation the image copies can be reflective of the sensation, e.g., a picture or video of mud, a bath tub of water or a dish of food.

Non-limiting example embodiments of the auditory stimulus presented in step 115 include presenting one or more of audio copies of music, talk, or other sounds produced in a time period corresponding to the earlier experience. For instance, when the earlier experience corresponds to an earlier era in the subject's life, the audio copies can be from publically available records, and/or the subject's personal recordings, of radio, movie or television programs, or home audio recordings, produced during the earlier era. For instance, when the earlier experience corresponds to an earlier sensation the audio copies can be reflective of the sensation, e.g., the sounds associated with touching mud, entering a bath tub and taking a bath, or preparing or eating a dish of food.

In some embodiments, the visual and auditory stimuli can be integrated into a single presentation e.g., a recording of a television program or movie, or, a home audio/video recording from the earlier era or the earlier sensation.

In some embodiments, the method 100, can further include pre-selecting the earlier experience (step 150), e.g., selecting before the stimulating step 102. In some embodiments, the subject can pre-select the earlier experience based on their personnel recollection of the earlier era, or earlier sensation, and their associated feelings of vigor.

In some embodiments, the earlier experience can be pre-selected for the subject based on the subject's demographics, e.g., age, sex, birthplace, present or previous location of habitation, cultural background, schooling, occupation, etc. . . .

In some embodiments, the auditory stimuli can include, and in some embodiments, consist of, music deemed to be relaxing, followed by music associated with the time period of the earlier time to be experienced. In some such embodiments, concurrently, visual abstract art intended to enhance the hypnogogic state and/or an additional audio verbal guidance audio track can be overlayed on the relaxation and/or earlier time period music tracks such that verbal guidance is provided to visualize memories stimulating human senses (e.g., verbal guidance to remember smells of perfumes or other smells from the earlier time to be experience).

In, some embodiments, the presenting of the visual stimulus includes presenting one or more of abstract graphics with additional audio or verbal guidance to visualize memories of the earlier experience to thereby stimulate the recollection of one or more of audio, visual or olfactory senses.

As a non-limiting example, the subject may be offered a menu to choose from a one of a matrix of timeframes and culture centers pairing. The timeframe selection could, e.g., support selections from among 7 different decades of time. The cultural selection could, e.g., support selections from among 10 different cultural centers within the USA, e.g., 5 across latitudes and each of those 5 divided by a northern and a southern cultural experience. Based on the pre-selection, a particular combination of visual and audio stimuli reflective of the selected timeframe and culture center could be loaded (e.g., uploaded from a database) for presentation to the subject as part of steps 110 and 115. In some embodiments, the subject could be allowed to select a particular combination of visual and audio stimuli by selecting specific image and audio recording from a menu of stock choices (e.g., a selection of specific pictures, videos or music reflective of the selected timeframe and culture center).

As noted above, we believe that, after stimulating the age regression experience, the subject will regain a degree of vigor as reflected by having one or more attributes of enhanced energy, confidence, or vitality, and/or, having one or more of improved physical or intellectual attributes as compared to these attributes measured before the stimulation.

For instance, as part of the method 100, the subject can be assessed for increases in vigor from the age regression experience, e.g., by comparing changes in one or more of attributes reflective of vigor (step 160) before (step 162) and after (step 164) stimulating the age regression experience in step 102. For instance, as part of steps 162 and 164, the subject can take a self-assessment survey, or be interviewed, to grade their subjective feelings of vigor (e.g., self-assessment grades of energy, confidence or vitality) before and after the stimulating step 102, respectively. For instance, as part of steps 162 and 164, the subject's physical or mental attributes (e.g., by measuring subject's strength, flexibility, balance, intelligence quotient, immunity functions or cognitive levels) can be objectively measured before and after the stimulating step 102, respectively. The difference or relative change in these subjective grades or objective measures can be compared as part of step 160.

In some embodiments it can be advantageous for the method 100 to include a step 170 to reduce the subject's exposure to other ambient visual and auditory stimuli during the stimulation step 102, e.g., so that the subject's attention is focused on the relaxation program and visual and auditory stimuli of steps 105, 110, 115 and 140 and is not otherwise distracted. For instance, as further explained below, some embodiments of the apparatus 200 can include features to block or reduce ambient visual and auditory stimuli.

Another embodiment of the disclosure is an apparatus, e.g., to implement embodiments of the method. With continuing reference to FIG. 1, as illustrated in FIG. 2, the apparatus 200 comprises an audio-video module 205 to simulate an age regression experience in a human subject (e.g., step 102). The audio-video module 205 includes an audio component 210 to present for the subject's attention, an audio relaxation program to induce a hypnogogic state in the subject (e.g., step 105), and then while the subject is in the hypnogogic state, present for the subject's attention, an auditory stimulus corresponding to an earlier experience in the subject's life (e.g., step 115). The audio-video module 205 also includes a video component 215 to present for the subject's attention, a visual stimulus corresponding to the earlier experience (e.g., step 110).

In some embodiments, the video component 215 can further present a video relaxation program to help induce the hypnogogic state (e.g., step 140).

In some embodiments, the audio-video module 205 can further include a mixer module 220 (e.g., an audio-video mixer or switcher) that is able to select one or more different video and audio sources and provide a composite mixture of such sources. For instance the mixer module is able to adjust relative volumes of the audio relaxation program and the audio stimulus during a transition period (e.g., step 145) from the presenting of the audio relaxation program to the presenting of the audio stimulus, where the two video sources and two audio source are faded out and faded as discussed in the context of step 145.

In some embodiments, the audio component 210 includes an audio device that includes sound mufflers 230 to block ambient audio stimuli (e.g., as part of step 170) from reaching the subject's attention during the presenting of the audio relaxation program and the presenting of the audio stimulus (e.g., step 115).

In some embodiments, the video component 215 includes one or more light shields 232 surrounding a video display 235 of the video component 215. The light shields 232 can be positioned to block ambient visual stimuli (e.g., as part of step 170) from reaching the subject's attention during the presenting of the visual stimulus (e.g., step 110).

For instance, in some embodiments the audio component 210 can be or include one or more speakers positioned to present one or more audio recording of the audio relaxation program and sound recordings reflective of the earlier experience, and, the video component can be or include an electronic display screen (e.g., CRT, LCD, DLP displays) configured to present one or more video recording of the video relaxation program and/or video recordings reflective of the earlier experience. In some such embodiments, both the audio and video components 210, 215 can be combined in a virtual reality headset to provide the audio-video module 205. For instance, the module 205 as a virtual reality headset can be shaped as goggles with the light shields 232 surrounding the view screen 235 to block ambient visual stimuli, and headphones with insulating mufflers to block ambient audio stimuli.

Alternatively or additionally to help reduce the subject's exposure to other ambient visual and auditory stimuli (e.g., step 170) during the stimulation step 102 the apparatus 200 can further include a sensory restriction chamber 250. For instance, embodiments of the chamber 250 can include a support structure 252 (e.g., a chair, a recliner, or a mattresses, such as a temperature regulated water bed mattresses with a soft covering material, such as a sheep-skin covering) located inside the chamber. The support structure 252 is sized to support the subject thereon during the stimulating of the age regression experience (e.g., step 102). Embodiments of the chamber 250 can include sound insulation tiles 254 covering substantially the entire outer surface 256 of the chamber 250. Embodiments of the chamber 250 can include a light-absorbing coating 258 (e.g., black paint, paper, fabric or plastic sheets) that covers the inner surface 260 of the chamber 250. As further illustrated the chamber 250 can include a ventilation unit 262, and include one or more shelves 264, e.g., on interior wall on which equipment, such as the audio-video module 205, can be stored, and a door 266 to facilitate access to the chamber 250.

Some embodiments of the apparatus 200 can further include a selection module 270 (e.g., touch-screen sensitive computer or such as a computer or phone tablet) to allow the subject to pre-select the earlier experience from a menu of choices (e.g., step 150), or to allow the subject to perform various self-assessment surveys (e.g., steps 122, 124, 130, 162, 164).

Some embodiments of the apparatus 200 can further include an EEG module 272, to measure the subject's brain wave activity (e.g., step 132) during the simulating (step 102).

Some embodiments of the apparatus 200 can further include a database module 275 (e.g., a hard drive or other electronic storage media accessible by a computer). The database module 275 can hold digital recordings of: the audio and video relaxation programs; the audio and video stimuli indexed into categories according to different combinations of timeframe and culture center, or surveys to assess the subject's the earlier experience, the hypnogogic state, or state of vigor.

Some embodiments of the apparatus 200 can further include a control module 280 (e.g., a CPU of a computer 282 with the database module 275 or with access the database module 275 stored at a remote location such as a server). The control module 280 can connect to the database module 275 and download to the audio-video module 205 and/or the mixer module 220, digital recordings of the audio and video stimuli corresponding to the indexed category corresponding to the earlier experience that was pre-selected by the subject (e.g., via the selection module 270). The audio-video module 205, mixer module 220, selection module 270, EEG module 272, database module 275, and, control module 280 can all be in data communication with each other via wired or wireless telecommunication procedures and equipment familiar one skilled in the pertinent art. In some embodiments, the audio-video module 205, mixer module 220, selection module 270, EEG module 272, database module 275, and, control module 280 can all be in a same location as the subject and the optional chamber 250, while in other embodiments one or more of the mixer module 220, selection module 270, EEG module 272, database module 275, and, control module 280, or a portion of the module, can be in a location remote from the subject the audio-video module 205 and the optional chamber 250.

Those skilled in the art to which this application relates will appreciate that other and further additions, deletions, substitutions and modifications may be made to the described embodiments.

1. A method, comprising:
stimulating an age regression experience in a human subject, including:
inducing a hypnogogic state in the subject by presenting, using audio-video equipment, for the subject's attention, an audio relaxation program; and
presenting, using the audio-video equipment, for the subject's attention, a visual stimulus and an audio stimulus corresponding to an earlier experience of a recollected memory of a feeling or a sensation from an earlier time in the subject's life

2. The method of claim 1, further including assessing by completing a survey presented on a computer or phone an extent of the subject's age regression experience during the stimulating.

3. The method of claim 1, further including assessing by measuring the subject's brain wave activity using electroencephalogram equipment, a presence of the hypnogogic state in the subject during the stimulating.

4. The method of claim 1, wherein the stimulating further includes presenting, using the audio-video equipment, a video relaxation program to induce the hypnogogic state.

5. The method of claim 1, wherein the stimulating further includes a transitional period between the presenting of the audio relaxation program and the presenting of the audio stimulus wherein, using an audio mixer of the audio-video equipment, a volume of the audio relaxation program is gradually decreased to become inaudible and a volume of the audio stimulus is gradually increased to become audible to the subject.

6. The method of claim 1, wherein the stimulating further includes presenting, using the audio-video equipment a video relaxation program with a transitional period between the presenting of the video relaxation program and the presenting of the visual stimulus, wherein, using a video mixer of the audio-video equipment, the video relaxation program is gradually faded out to become non-visible to the subject and the visual stimulus is gradually faded in to become visible to the subject.

7. The method of claim 1, wherein the recollected memory of the feeling is the subject's recollection of having a feeling of vigor during the earlier time as measured from the subject's completion of a survey presented on a computer or phone.

8. The method of claim 1, wherein the recollected memory of the sensation is the subject's recollection having one or more of: a feeling of mud, a warm bath, a smell of food or a taste of food during the earlier time.

9. The method of claim 1, wherein the presenting of the visual stimulus using the audio-video equipment includes presenting one of more of image copies of pictures, drawings, photographs or video recordings produced in a time period corresponding to the earlier experience.

10. The method of claim 1, wherein the presenting of the visual stimulus using the audio-video equipment includes presenting one or more of abstract graphics with additional audio or verbal guidance to visualize memories of the earlier experience to thereby stimulate the recollected memory of one or more of audio, visual or olfactory senses.

11. The method of claim 1, wherein the presenting of the auditory stimulus using the audio-video equipment includes presenting one or more of audio copies of music, talk, or other sounds produced in a time period corresponding to the earlier experience.

12. The method of claim 1, further including having the subject pre-select the earlier experience from a menu of choices of images of one or more of pictures, drawings, photographs or video recordings or audio copies of one or more music, talk, or other sounds, corresponding to the earlier experience and stored on a computer or a phone.

13. The method of claim 7, further including assessing by comparing the survey results presented on a computer or phone to measure a change in an extent of a grading, stored on an electronic storage media of the computer or the phone, of the subject's feeling of the vigor from the age regression experience.

14. The method of claim 1, further including reducing the subject's exposure to ambient visual and auditory stimuli during the stimulating of the age regression experience by placing the subject into a chamber that blocks exposure to the ambient visual and auditory stimuli.

15-21. (canceled)
